(19) United States
(12) Patent Application Publication
(10) Pub. No.: US 2025/0325765 A1
(43) Pub. Date: Oct. 23, 2025

(54) NON-INVASIVE JET VENTILATOR PATIENT INTERFACE WITH MASS FLOW MEASUREMENT

(71) Applicant: HILL-ROM SERVICES PTE. LTD., SINGAPORE (SG)

(72) Inventor: Gary J. LaTorraca, Huntington Beach, CA (US)

(21) Appl. No.: 19/175,217

(22) Filed: Apr. 10, 2025

Related U.S. Application Data
(60) Provisional application No. 63/637,603, filed on Apr. 23, 2024.

(51) Int. Cl. A61M 16/00; A61M 16/06; A61M 16/08; A61M 16/10

(52) U.S. Cl. CPC A61M 16/0096; A61M 16/0622; A61M 16/0666; A61M 16/0858; A61M 16/1045; A61M 2016/0027; A61M 2202/0208; A61M 2205/42

(57) ABSTRACT

A patient ventilation interface comprises a jet nozzle and a throat body that is arranged to receive ventilation gas output by the jet nozzle. The throat body defines a gas inlet and a gas outlet, with the gas inlet being open to ambient air. The patient ventilation interface further comprises a first pressure sensing tube having a pressure sensing port positioned within the throat body downstream of the jet nozzle and a second pressure sensing tube having a pressure sensing port positioned within the throat body downstream of the pressure sensing port of the first pressure sensing tube. A controller may be configured to calculate a mass flow based on pressure measurements taken at the pressure sensing ports of the first and second pressure sensing tubes.

### CROSS-REFERENCE TO RELATED APPLICATIONS

The present application claims priority to U.S. Provisional Application Ser. No. 63/637,603 filed Apr. 23, 2024, the disclosure of which is incorporated herein by reference.

### STATEMENT RE: FEDERALLY SPONSORED RESEARCH/DEVELOPMENT

Not Applicable

### BACKGROUND

### 1. Technical Field

The present disclosure relates generally to devices and methods for the administration of non-invasive ventilation (NIV) therapy and, more particularly, to an improved patient ventilation interface.

### 2. Description of the Related Art

Comfort and ergonomics are important aspects of non-invasive ventilation, and key determinants to acceptance of therapy and compliance thereto. Extant solutions generally fall into two categories. One involves a stationary device with large diameter tubing and patient interface, commonly a mask. These systems confine the patient to a fixed location with a bulky apparatus that limits movement and mobility. The other solution involves a jet-based interface that uses small diameter tubing connected to a small and mobile device. These systems tend to be louder than the mask-based system, lack optimal humidification, and lack the capability to measure tidal volume. Both solutions are inefficient positive end-expiratory pressure (PEEP) generators and are thus wasteful of patient gas.

### BRIEF SUMMARY

The present disclosure contemplates various systems and methods for overcoming the above drawbacks accompanying the related art. One aspect of the embodiments of the present disclosure is a patient ventilation interface. The patient ventilation interface may comprise a jet nozzle and a throat body arranged to receive ventilation gas output by the jet nozzle. The throat body may define a gas inlet and a gas outlet, the gas inlet being open to ambient air. The patient ventilation interface may further comprise a first pressure sensing tube having a pressure sensing port positioned within the throat body downstream of the jet nozzle and a second pressure sensing tube having a pressure sensing port positioned within the throat body downstream of the pressure sensing port of the first pressure sensing tube.

The patient ventilation interface may comprise a throat housing containing the throat body. The throat housing may define a plenum between an outer wall of the throat body and an inner wall of the throat housing. The patient ventilation interface may comprise a pilot pressure line in fluid communication with the plenum. The patient ventilation interface may comprise a pair of nasal pillows downstream of the throat body and arranged to receive a combined flow of gas from the gas outlet of the throat body, the combined flow of gas including the ventilation gas received by the throat body from the jet nozzle and entrained ambient air received by the throat body via the gas inlet. The patient ventilation interface may comprise a supplemental oxygen tube arranged to provide supplemental oxygen gas outside the gas inlet. The combined flow of gas received by the throat body may include the supplemental oxygen gas. The patient ventilation interface may comprise a heat and moisture exchanger (HME) downstream of the gas outlet of the throat body and upstream of the pair of nasal pillows. The pair of nasal pillows may be arranged to receive the combined flow of gas via the HME. The patient ventilation interface may comprise a ventilation gas tube that terminates in the jet nozzle. The ventilation gas tube may define a main flow lumen for the ventilation gas, a first sense lumen in fluid communication with the first pressure sensing tube, and a second sense lumen in fluid communication with the second pressure sensing tube. The patient ventilation interface may comprise a muffler surrounding the gas inlet of the throat body. The muffler may comprise a cylindrical sleeve.

Another aspect of the embodiments of the present disclosure is a patient ventilation system. The patient ventilation system may comprise a jet nozzle and a throat body arranged to receive ventilation gas output by the jet nozzle. The throat body may define a gas inlet and a gas outlet, the gas inlet being open to ambient air. The patient ventilation system may further comprise a first pressure sensing tube having a pressure sensing port positioned within the throat body downstream of the jet nozzle, a second pressure sensing tube having a pressure sensing port positioned within the throat body downstream of the pressure sensing port of the first pressure sensing tube, a first pressure sensor in fluid communication with the first pressure sensing tube, a second pressure sensor in fluid communication with the second pressure sensing tube, and a controller configured to calculate a mass flow based on a first pressure measurement taken by the first pressure sensor and a second pressure measurement taken by the second pressure sensor.

The patient ventilation system may comprise a throat housing containing the throat body. The throat housing may define a plenum between an outer wall of the throat body and an inner wall of the throat housing. The patient ventilation system may comprise a pilot pressure line in fluid communication with the plenum. The controller may be configured to calculate the mass flow further based on a pressurization state of the plenum. The controller may be configured to control delivery of the ventilation gas by the jet nozzle based on the second pressure measurement taken by the second pressure sensor. The patient ventilation system may comprise a pair of nasal pillows downstream of the throat body and arranged to receive a combined flow of gas from the gas outlet of the throat body, the combined flow of gas including the ventilation gas received by the throat body from the jet nozzle and entrained ambient air received by the throat body via the gas inlet. The patient ventilation system may comprise a supplemental oxygen tube arranged to provide supplemental oxygen gas outside the gas inlet. The combined flow of gas received by the throat body may further include the supplemental oxygen gas. The patient ventilation system may comprise a heat and moisture exchanger (HME) downstream of the gas outlet of the throat body and upstream of the pair of nasal pillows, the pair of nasal pillows being arranged to receive the combined flow of gas via the HME. The patient ventilation system may comprise a ventilation gas tube that terminates in the jet nozzle, the ventilation gas tube defining a main flow lumen for the ventilation gas, a first sense lumen in fluid communication with the first pressure sensing tube, and a second sense lumen in fluid communication with the second pressure sensing tube. The patient ventilation system may comprise a muffler surrounding the gas inlet of the throat body. The muffler may comprise a cylindrical sleeve.

Another aspect of the embodiments of the present disclosure is a method of determining mass flow in a patient ventilation interface including a jet nozzle and a throat body that is arranged to receive ventilation gas output by the jet nozzle and defines a gas inlet and a gas outlet, the gas inlet being open to ambient air. The method may comprise measuring a first pressure at a first position within the throat body downstream of the jet nozzle, measuring a second pressure at a second position within the throat body downstream of the first position, and calculating a mass flow based on the measured first and second pressures.

The mass flow may be calculated further based on a pressurization state of a plenum defined between an outer wall of the throat body and an inner wall of a throat housing that contains the throat body. A patient ventilation method may comprise the described method of determining mass flow in the patient ventilation interface and may further comprise controlling delivery of the ventilation gas by the jet nozzle based at least in part on the calculated mass flow.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 shows a system including an exemplary patient ventilation interface according to an embodiment of the present disclosure;

FIG. 2 is another view of the system including a ventilator thereof together with a cross-sectional view of the patient ventilation interface; and

FIG. 3 is another view of the system including a cross-sectional view of the patient ventilation interface.

### DETAILED DESCRIPTION

The present disclosure encompasses various embodiments of a patient ventilation interface for use in a non-invasive ventilation system, along with systems and methods for mass flow measurement using the patient ventilation interface. The detailed description set forth below in connection with the appended drawings is intended as a description of several currently contemplated embodiments and is not intended to represent the only form in which the disclosed interface may be developed or utilized. The description sets forth the functions and features in connection with the illustrated embodiments. It is to be understood, however, that the same or equivalent functions may be accomplished by different embodiments that are also intended to be encompassed within the scope of the present disclosure. It is further understood that the use of relational terms such as first and second and the like are used solely to distinguish one from another entity without necessarily requiring or implying any actual such relationship or order between such entities.

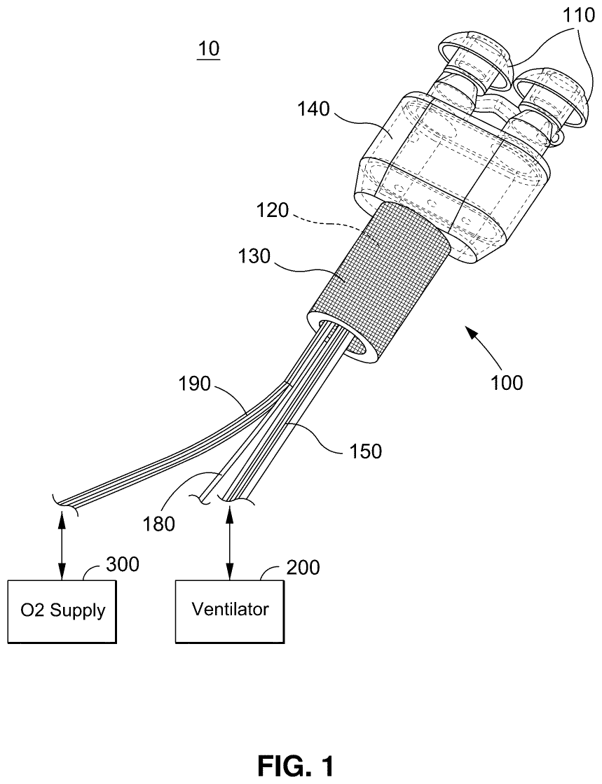

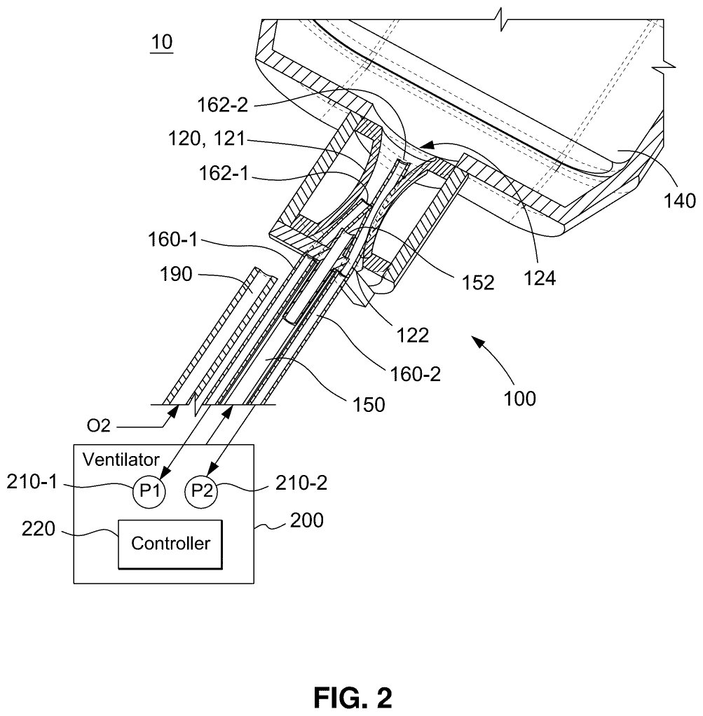

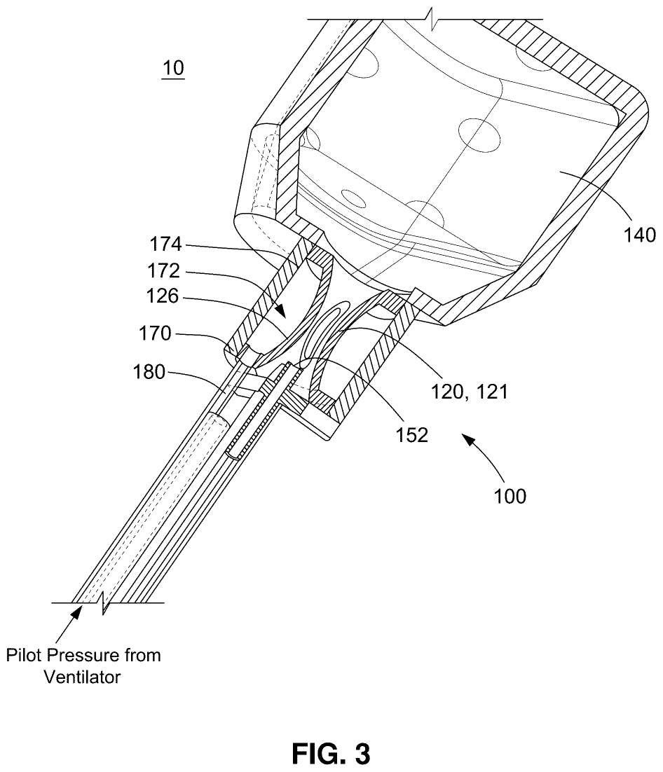

FIG. 1 shows a system 10 including an exemplary patient ventilation interface 100 according to an embodiment of the present disclosure. FIGS. 2 and 3 are additional views of the system 10 showing cross-sectional views of the patient ventilation interface 100. As illustrated, the patient ventilation interface 100 may be of a nasal pillows type defined by a pair of nasal pillows 110 that are configured to be inserted at least partially into the patient's nares or nostrils. Except as provided herein, the nasal pillows 110 may, for example, be the same as those described in U.S. Patent Application Pub. No. 2022/0339378, filed Apr. 4, 2022 and entitled “Accurate Pressure Measurement with Non-Invasive Ventilation Nasal Pillows,” the entire contents of which is incorporated by reference herein. Delivery of ventilation gas to the patient may be achieved via a jet venturi 120 (see FIG. 2), which may be surrounded by a muffler 130 and therefore not visible in FIG. 1. The jet venturi 120 may be fluidly coupled to the nasal pillows 110 via a heat and moisture exchanger (HME) 140. The ventilation gas to be delivered to the patient by the jet venturi 120 may be provided by a ventilator 200 of the system 10 via a ventilation gas tube 150, which may be one of several tubes (or lumens within a multi-lumen tube) as described in more detail below.

Referring to FIG. 2, the jet venturi 120 may comprise a throat body 121 that is arranged to receive ventilation gas output by a jet nozzle 152. In this regard, the ventilation gas may be provided to the throat body 121 from the ventilator 200 via the ventilation gas tube 150, which may terminate in the jet nozzle 152 as shown. The throat body 121 may define a gas inlet 122 and a gas outlet 124, with the gas inlet being open to ambient air. With the arrangement shown in FIG. 2, the throat body 121 may receive the ventilation gas output by the jet nozzle 152 via the gas inlet 122, in addition to ambient air which is drawn through the gas inlet 122 and likewise introduced into the throat body 121. The distal tip of the jet nozzle 152 may be either inside the throat body 121 as shown (i.e., downstream of the gas inlet 122) or outside the throat body 121 (i.e., upstream of the gas inlet 122), with the entrainment of ambient air occurring around the periphery of the jet nozzle 152 in either case. Due to the increased velocity of the ventilation gas at a constriction of the throat body 121 between the gas inlet 122 and the gas outlet 124, there is a decrease in pressure that causes ambient air to be entrained via the gas inlet 122 (by operation of the venturi effect). By amplifying the ventilation gas output by a jet nozzle 152 in this way, the jet venturi 120 may serve as an efficient flow generator when providing ventilation therapy to the patient. Because the amplification of the ventilation gas may occur at the jet venturi 120, it is noted that the nasal pillows 110 themselves need not include any entrainment openings.

In order to support direct measurement of patient tidal volume, the patient ventilation interface 100 may advantageously incorporate a pair of pressure taps arranged to enable manometer-type mass flow sensor measurement. As shown in FIG. 2 by way of example, the patient ventilation interface 100 may include a first pressure sensing tube 160-1 having a pressure sensing port 162-1 positioned within the throat body 121 downstream of the jet nozzle 152 and a second pressure sensing tube 160-2 having a pressure sensing port 162-2 positioned within the throat body 121 downstream of the pressure sensing port 162-1 of the first pressure sensing tube 160-1. Pressure measurements associated with the first and second pressure sensing ports 162-1, 162-2 may be taken respectively by a first pressure sensor 210-1 in fluid communication with the first pressure sensing tube 160-1 and a second pressure sensor 210-1 in fluid communication with the second pressure sensing tube 160-2. A controller 220 may be configured to calculate a mass flow based on a first pressure measurement taken by the first pressure sensor 210-1 and a second pressure measurement taken by the second pressure sensor 210-2. Delivery of the ventilation gas by the jet nozzle 152 may then be controlled based at least in part on the calculated mass flow. For example, under programmatic control, the ventilator 200 may deliver a breath meant to realize a flow or pressure vs. time waveform. The target waveform may be predetermined, e.g. square, descending ramp, or other. It might also be generated on the fly to respond to patient effort. Regardless of the intended delivery waveform, the measured mass flow may be fed back for inclusion in the calculations leading to the next control output. As represented in FIG. 2, the controller 220, as well as the pressure sensors 210-1, 210-2 may be provided in the ventilator 200.

To measure delivered tidal volume and exhaled volume, the pressure difference between the first and second pressure sensing ports 162-1, 162-2 can be characterized and calibrated to measure mass flow through the throat body 121. For example, a Riemann sum of sampled mass flow, e.g., at 5 ms intervals, in combination with inspiration and exhalation detection algorithms (e.g., based on measured airway pressure at the second pressure sensing port 162-2) can deliver accurate tidal volume and exhaled volume calculations. Pressure difference between the first and second pressure sensing ports 162-1, 162-2 vs. mass flow through the throat is a function of the throat area. As such, the pressure sensing ports 162-1, 162-2 may be arranged far enough apart from each other in order to ensure an appreciable difference in measured pressure. For example, the second pressure sensing port 162-2 may be arranged at or near the gas outlet 124 where the throat body 121 may be widest, in order to most closely represent airway pressure of the patient for purposes of breath control. In this case, the first pressure sensing port 162-1 may be arranged at or near the constriction where the throat body 121 is narrowest. The known cross-sectional area of the throat body 121 at these two positions may be referenced by the controller 220 to relate first and second pressure measurements to mass flow. In this way, the total flow to the patient may be directly measured rather than being estimated as described, for example, in U.S. Pat. No. 11,607,519 (“the '519 patent”), issued Mar. 21, 2023 and entitled “O₂ Concentrator with Sieve Bed Bypass and Control Method Thereof,” the entire contents of which are incorporated by reference herein.

So that the jet venturi 120 may serve as an efficient PEEP generator, the throat body 121 may be deformable to enable different states of constriction, for example, as described in U.S. Patent Application Pub. No. 2022/0249797, filed Dec. 28, 2021 and entitled “Variable Throat Jet Venturi,” the entire contents of which are incorporated by reference herein. In this respect, with reference to FIG. 3, the patient ventilation interface 100 may include a throat housing 170 containing the throat body 121. The throat housing 170 may define a plenum 172 between an outer wall 126 of the throat body 121 and an inner wall 174 of the throat housing 170. The patient ventilation interface 100 may include a pilot pressure line 180 in fluid communication with the plenum 172. The pilot pressure line 180 may be fluidly coupled to a pneumatic drive circuit in the ventilator 200, for example. Under programmatic control, and using airway pressure feedback (e.g., from the second pressure sensing port 162-2), the plenum 172 may be pressurized to effect different throat cross-sectional areas. By selectively pressurizing the plenum 172, the plenum 172 can be transitioned between a first pressurization state for maximizing airflow to the patient (e.g. during inhalation) and a second pressurization state in which the deformable throat body 121 is more constricted. In the latter state, the reduced cross-sectional area of the deformable throat body 121 may significantly reduce the required nozzle flow for achieving a desired output pressure, making it possible to efficiently generate PEEP with minimal gas consumption (and reduced audible sound due to the reduced gas flow from the jet nozzle 152). At zero drive pressure the deformable throat body 121 may be in its free state and its diameter may be maximum, minimizing exhalation resistance for the patient in a zero PEEP condition.

As noted above, the controller 220 may be configured to calculate a mass flow based on the pressure measurements taken at the first and second pressure sensing ports 162-1, 162-2 by referencing the known cross-sectional area of the throat body 121 at these two positions. Due to the variable throat area in embodiments having a deformable throat body 121 as described, the cross-sectional area of the throat body 121 may depend on the pressurization state of the plenum 172. As such, it is contemplated that the controller 220 may be configured to calculate the mass flow further based on a pressurization state of the plenum 172. For example, the controller 220 may reference a plurality of known cross-sectional areas of the throat body 121 at the two positions of the first and second pressure sensing ports 162-1, 162-2, with the known cross-sectional areas being indexed by pressurization state (e.g., pressure value of the pilot pressure line 180). For a given pressurization state, the corresponding cross-sectional areas of the throat body 121 at the positions of the first and second pressure sensing ports 162-1, 162-2 may then be used to relate the pressure measurements to mass flow.

As noted above, the jet venturi 120 comprising the throat body 121 may be fluidly coupled to the nasal pillows 110 via a heat and moisture exchanger (HME) 140. Exhaled patient gas is typically saturated with water vapor at or near body temperature. In the disclosed patient ventilation interface 100, the exhaled gas may travel from the patient through the nasal pillows 110 and through the HME 140 as it exits the interface to the ambient environment (e.g., via the gas inlet 122 of the throat body 120). The inline HME 140 may absorb a significant amount of water vapor during exhalation that may subsequently be evaporated and redelivered to the patient during the next inspiration. In this way, patient comfort may be maintained and minimum humidity levels as defined by ISO 80601-2-74 Humidifier Particular Standard may be achieved. When the HME 140 needs to be replaced, a housing containing the HME 140 may be opened (e.g., by a clamshell opening) so that the HME 140 may be easily swappable.

In general, audible noise is inherent in the operation of a gas jet venturi. The working fluid (e.g., air or an air/oxygen mixture), undergoes shear through the throat and generates pressure waves in the audible range. In view of minimizing this noise, the disclosed patient ventilation interface 100 may incorporate a muffler 130 as shown in FIG. 1. The muffler 130 may comprise a cylindrical sleeve (e.g., made of a non-rigid and pliable material such as silicon rubber foam) that surrounds at least the intake/inlet side of the jet venturi 120, namely, the gas inlet 122 of the throat body 121 in the illustrated example shown in FIGS. 1 and 2. Audible noise that is reflected back from the throat body 121 toward the ambient environment may advantageously be attenuated by the muffler 130.

As best shown in FIG. 2, the ventilation gas tube 150 may terminate in the jet nozzle 152. The ventilation gas tube 150 may define a main flow lumen for the ventilation gas and may further define one or more additional lumens. For example, the ventilation gas tube 150 may define, in addition to a main flow lumen for the ventilation gas, a first sense lumen in fluid communication with the first pressure sensing tube 160-1 and a second sense lumen in fluid communication with the second pressure sensing tube 160-2. The first and second pressure sensing tubes 160-1, 160-2 may extend from these first and second sense lumens, respectively, into the throat body 121 as shown in FIG. 2 (the lumens are not separately referenced). As an alternative to the multi-lumen tube construction, the ventilation gas tube 150 may instead have a tube-within-a-tube construction in which the first and second pressure sensing tubes 160-1, 160-2 simply reside within the larger ventilation gas tube 150. As a further alternative, the ventilation gas tube 150 and pressure sensing tubes 160-1, 160-2 may be entirely separate tubes that are bundled together. While the pilot pressure line 180 is illustrated separately in FIG. 1, it is also contemplated that the pilot pressure line 180 may likewise be included with the ventilation gas tube 150 and pressure sensing tubes 160-1, 160-2, either as part of a multi-lumen or tube-within-a-tube construction or as part of a bundle of tubes.

As shown in FIGS. 1 and 2, the patient ventilation interface 190 may additionally include a supplemental oxygen tube 190 that may be arranged to provide supplemental oxygen gas outside the gas inlet 122. The supplemental oxygen tube 190 may terminate near enough to the gas inlet 122 so that the supplemental oxygen gas is entrained along with the ambient air by operation of the venturi effect (as described above). Because the supplemental oxygen tube 190 may typically be connected to an O₂ supply 300 (e.g., an oxygen tank or oxygen concentrator) that is separate from the ventilator 200, it is contemplated that the supplemental oxygen tube 190 may be separate from the ventilation gas tube 150, pressure sensing tubes 160-1, 160-2, and pilot pressure line 180 (i.e., not part of a multi-lumen or tube-within-a-tube construction) but may be at least partially bundled with the other tubes by retention means such as a plastic clip piece or tube retention feature molded into the muffler 130. Typically, 25% of the total gas delivered to a patient by a jet venturi NIV system such as the system 10 is ventilation gas output by the ventilator, which may have an oxygen gas concentration in the range of 21%-100%. In this regard, an example ventilator 200 that may deliver a variable range of oxygen gas concentration is a ventilator driven by a portable gas source (PGS) comprising an oxygen concentrator as described in the '519 patent. Typically, 75% of the delivered flow is entrained from the ambient environment, e.g. room air at 21% O₂, limiting the fraction of inspired oxygen (FiO₂) that can be achieved in this manner. By including the supplemental oxygen tube 190, FiO₂ can be increased by directing concentrated oxygen (85%-95% O₂) from the O₂ supply 300 to the gas inlet 122 of the throat body 121 to be entrained together with (or instead of) the ambient air.

The controller 220 of the patient ventilation system 10 (which may be a controller of the ventilator 200 but may also be a controller of an oxygen concentrator or PGS or a standalone device) may be implemented with a programmable integrated circuit device such as a microcontroller or control processor. Broadly, the device may receive certain inputs, and based upon those inputs, may generate certain outputs. The specific operations that are performed on the inputs may be programmed as instructions that are executed by the control processor. In this regard, the device may include an arithmetic/logic unit (ALU), various registers, and input/output ports. External memory such as EEPROM (electrically erasable/programmable read only memory) may be connected to the device for permanent storage and retrieval of program instructions, and there may also be an internal random-access memory (RAM). Computer programs for implementing any of the disclosed functionality of the controller 220 may reside on such non-transitory program storage media, as well as on removable non-transitory program storage media such as a semiconductor memory (e.g. IC card), for example, in the case of providing an update to an existing device. Examples of program instructions stored on a program storage medium or computer-readable medium may include, in addition to code executable by a processor, state information for execution by programmable circuitry such as a field-programmable gate arrays (FPGA) or programmable logic device (PLD).

The various features of the disclosed patient ventilation interface 100 and system 10 may be implemented separately or in various combinations to achieve the functions described herein. In combination, the features and functions of the disclosed patient ventilation interface 100 and system 10 may afford an interface that is small, comfortable, and ergonomic, enabling mobility during NIV while providing humidification, flow measurement, sound attenuation, and supplemental oxygen delivery. The patient ventilation interface 100 may be configured in a bolo tie style in which the tubing is arranged to extend downward from the patient ventilation interface 100 in front of the patient's face (e.g., as shown in FIG. 1) or as an overhead interface in which the tubing extends outward and upward to the top of the patient's head.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A patient ventilation interface comprising:
a jet nozzle;
a throat body arranged to receive ventilation gas output by the jet nozzle and defining a gas inlet and a gas outlet, the gas inlet being open to ambient air;
a first pressure sensing tube having a pressure sensing port positioned within the throat body downstream of the jet nozzle; and
a second pressure sensing tube having a pressure sensing port positioned within the throat body downstream of the pressure sensing port of the first pressure sensing tube.

2. The patient ventilation interface of claim 1, further comprising:
a throat housing containing the throat body, the throat housing defining a plenum between an outer wall of the throat body and an inner wall of the throat housing; and
a pilot pressure line in fluid communication with the plenum.

3. The patient ventilation interface of claim 1, further comprising a pair of nasal pillows downstream of the throat body and arranged to receive a combined flow of gas from the gas outlet of the throat body, the combined flow of gas including the ventilation gas received by the throat body from the jet nozzle and entrained ambient air received by the throat body via the gas inlet.

4. The patient ventilation interface of claim 3, further comprising a supplemental oxygen tube arranged to provide supplemental oxygen gas outside the gas inlet, the combined flow of gas received by the throat body further including the supplemental oxygen gas.

5. The patient ventilation interface of claim 3, further comprising a heat and moisture exchanger (HME) downstream of the gas outlet of the throat body and upstream of the pair of nasal pillows, the pair of nasal pillows being arranged to receive the combined flow of gas via the HME.

6. The patient ventilation interface of claim 1, further comprising a ventilation gas tube that terminates in the jet nozzle, the ventilation gas tube defining a main flow lumen for the ventilation gas, a first sense lumen in fluid communication with the first pressure sensing tube, and a second sense lumen in fluid communication with the second pressure sensing tube.

7. The patient ventilation interface of claim 1, further comprising a muffler surrounding the gas inlet of the throat body.

8. The patient ventilation interface of claim 7, wherein the muffler comprises a cylindrical sleeve.

9. A patient ventilation system comprising:
a jet nozzle;
a throat body arranged to receive ventilation gas output by the jet nozzle and defining a gas inlet and a gas outlet, the gas inlet being open to ambient air;
a first pressure sensing tube having a pressure sensing port positioned within the throat body downstream of the jet nozzle;
a second pressure sensing tube having a pressure sensing port positioned within the throat body downstream of the pressure sensing port of the first pressure sensing tube;
a first pressure sensor in fluid communication with the first pressure sensing tube;
a second pressure sensor in fluid communication with the second pressure sensing tube; and
a controller configured to calculate a mass flow based on a first pressure measurement taken by the first pressure sensor and a second pressure measurement taken by the second pressure sensor.

10. The patient ventilation system of claim 9, further comprising:
a throat housing containing the throat body, the throat housing defining a plenum between an outer wall of the throat body and an inner wall of the throat housing; and
a pilot pressure line in fluid communication with the plenum,
wherein the controller is configured to calculate the mass flow further based on a pressurization state of the plenum.

11. The patient ventilation system of claim 10, wherein the controller is further configured to control delivery of the ventilation gas by the jet nozzle based on the second pressure measurement taken by the second pressure sensor.

12. The patient ventilation system of claim 9, further comprising a pair of nasal pillows downstream of the throat body and arranged to receive a combined flow of gas from the gas outlet of the throat body, the combined flow of gas including the ventilation gas received by the throat body from the jet nozzle and entrained ambient air received by the throat body via the gas inlet.

13. The patient ventilation system of claim 12, further comprising a supplemental oxygen tube arranged to provide supplemental oxygen gas outside the gas inlet, the combined flow of gas received by the throat body further including the supplemental oxygen gas.

14. The patient ventilation system of claim 12, further comprising a heat and moisture exchanger (HME) downstream of the gas outlet of the throat body and upstream of the pair of nasal pillows, the pair of nasal pillows being arranged to receive the combined flow of gas via the HME.

15. The patient ventilation system of claim 9, further comprising a ventilation gas tube that terminates in the jet nozzle, the ventilation gas tube defining a main flow lumen for the ventilation gas, a first sense lumen in fluid communication with the first pressure sensing tube, and a second sense lumen in fluid communication with the second pressure sensing tube.

16. The patient ventilation system of claim 9, further comprising a muffler surrounding the gas inlet of the throat body.

17. The patient ventilation system of claim 16, wherein the muffler comprises a cylindrical sleeve.

18. A method of determining mass flow in a patient ventilation interface including a jet nozzle and a throat body that is arranged to receive ventilation gas output by the jet nozzle and defines a gas inlet and a gas outlet, the gas inlet being open to ambient air, the method comprising:
measuring a first pressure at a first position within the throat body downstream of the jet nozzle;
measuring a second pressure at a second position within the throat body downstream of the first position; and
calculating a mass flow based on the measured first and second pressures.

19. The method of claim 18, wherein said calculating includes calculating the mass flow further based on a pressurization state of a plenum defined between an outer wall of the throat body and an inner wall of a throat housing that contains the throat body.

20. A patient ventilation method comprising:
the method of claim 18; and
controlling delivery of the ventilation gas by the jet nozzle based at least in part on the calculated mass flow.
